(19) United States
(12) Patent Application Publication
(10) Pub. No.: US 2020/0000539 A1
(43) Pub. Date: Jan. 2, 2020

(54) CONTROLLER FOR SURGICAL TOOLS

(71) Applicant: Human Xtensions Ltd., Netanya (IL)

(72) Inventors: Mordehai SHOLEV, Moshav Amikam (IL); Assaf KAUFMAN, Tal Shahar (IL)

(73) Assignee: Human Xtensions Ltd., Netanya (IL)

(21) Appl. No.: 16/487,870

(22) PCT Filed: Feb. 5, 2018
(86) PCT No.: PCT/IL2018/050127

§ 371 Date: Aug. 22, 2019

Related U.S. Application Data
(60) Provisional application No. 62/462,447, filed on Feb. 23, 2017.

(51) Int. Cl. A61B 34/00; A61B 1/00; A61B 17/29; A61B 90/53; A61B 1/313; B25J 13/02

(52) U.S. Cl. CPC A61B 34/74; A61B 1/00149; B25J 13/02; A61B 90/53; A61B 1/3132; A61B 17/29

(57) ABSTRACT

A controller for a surgical tool is provided. The controller includes an elongated body having first, second and third portions each movable with respect to each other. The controller further includes an interface engageable via a hand and/or fingers of a user.

### FIELD AND BACKGROUND OF THE INVENTION

The present invention relates to a controller for surgical tools and to methods of using same. Embodiments of the present invention relate to a controller for locally or remotely guiding and actuating one or more laparoscopic tools in a surgical procedure.

Minimally invasive procedures are performed through a small diameter access site in a tissue wall or through a natural orifice. Such procedures minimize trauma to tissue and organs and greatly reduce the patient's recovery period.

In endoscopic procedures performed through a tissue access site (e.g. laparoscopic procedures) a small incision is made in a tissue wall and a small cannula, termed a trocar, is inserted through the incision. The trocar defines a passageway through which various surgical tools (laparoscopes) can be inserted to perform cutting, suturing and removal of tissue.

In endoscopic procedures performed through a natural opening, an endoscope is inserted through the mouth, urethra, anus, etc. and guided to a tissue location in the GI tract, vaginal cavity or bladder to perform a diagnostic or surgical procedure. Endoscopic procedures also include Natural Orifice Transluminal Endoscopic Surgery (NOTES) in which an endoscopic tool is passed through the natural orifice and then through an internal incision in the stomach, vagina, bladder or colon, thus avoiding any external incisions or scars.

Endoscopic tools are guided within the body using an extracorporeal user controller which transfers hand/arm movement of the user to movement and actuation (collectively ‘operation’) of the surgical tool. Thus, the tool controller enables the user to control the operation of a surgical tool within the body from outside the body. Many types of tools can be controlled in this manner ranging grasper and scissor-like tools and cameras to complex robotic systems.

Numerous types of surgical tool controllers are known in the art, see for example, U.S. Pat. Nos. 7,996,110, 7,963,913, 8,521,331, 8,398,541, 8,939,891, 9,050,120 8,332,072, US20100170519, US20090036901, US20140222023, and US20140228631.

Commercially available robotic tool controllers such as the Da Vinci, TransEnterix and Titan systems are large and heavy and force the surgeon to sit in a console away from the patient bed. Such controllers are operated via a hand/finger levers or handles as well as foot pedals and require a high degree of coordination to smoothly operate the robotic surgical tools.

There is thus a need for a controller capable of remotely or locally controlling the operation of one or more surgical tools while being devoid of the abovementioned limitations of prior art controllers.

### SUMMARY OF THE INVENTION

According to one aspect of the present invention there is provided a controller for a surgical tool comprising an elongated body having: (a) a first portion having a proximal end attachable to a support and a distal end connected to a second portion through a first connector configured for enabling the second portion to move with respect to the first portion; and (b) a third portion connected to the second portion through a second connecter configured for enabling the third portion to move with respect to the second portion, the third portion having an interface engageable by a hand and/or fingers of a user.

According to further features in preferred embodiments of the invention described below, the proximal end of the first portion is movable with respect to the support when attached thereto.

According to still further features in the described preferred embodiment the first portion includes a first sensor for measuring a movement of the first portion with respect to the support.

According to still further features in the described preferred embodiment the elongated body includes a second sensor for measuring a movement of the second portion with respect to the first portion.

According to still further features in the described preferred embodiment the elongated body includes a third sensor for measuring a movement of the third portion with respect to the second portion.

According to still further features in the described preferred embodiment the elongated body is positioned under a forearm of a user when the interface of the third portion is engaged by hand and/or fingers of the user.

According to still further features in the described preferred embodiment the first portion is attachable to the support frame through a pivot.

According to still further features in the described preferred embodiment the first portion is capable of rolling and/or pivoting with respect to the support.

According to still further features in the described preferred embodiment the second portion is capable of translating and/or rolling with respect to the first portion.

According to still further features in the described preferred embodiment the third portion is capable of translating rolling and/or pivoting with respect to the second portion.

According to still further features in the described preferred embodiment the interface include levers engageable by a thumb and forefinger of the user.

According to still further features in the described preferred embodiment the second portion is engageable by a palm of the user.

According to still further features in the described preferred embodiment the controller further comprising a wireless transceiver for communicating with a surgical tool.

According to another aspect of the present invention there is provided a system comprising the controller attached to a surgical tool.

According to still further features in the described preferred embodiment the surgical tool is an endoscope.

According to still further features in the described preferred embodiment the surgical tool is steerable and includes an effector end.

According to still further features in the described preferred embodiment the effector end is a grasper, scissors, a needle, a camera, suction or a clamp.

The present invention successfully addresses the shortcomings of the presently known configurations by providing a surgical instrument controller having an easy to use interface that is natural to operate.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates the present controller operated by a hand of user.

FIG. 2 illustrates the relative motion of the three portions of the present controller.

FIGS. 3A-D illustrate the fore-aft of the present controller (FIGS. 3A, C) and the corresponding in and out (zoom) of a telescopic shaft of a surgical device (FIGS. 3B, D).

FIGS. 4A-B illustrate the left-right movement of the present controller (FIG. 4A) and the corresponding rotation of a shaft of a surgical device (FIG. 4B).

FIGS. 5A-B illustrate the up-down movement of the present controller (FIG. 5A) and the corresponding deflection of a steerable shaft of a surgical device (FIG. 5B).

FIGS. 6A-D illustrate the angular in-out movement of the finger levers of the present controller (FIGS. 6A, C) and the corresponding open-close jaw movement of a tissue manipulator end of a surgical device (FIGS. 6B, D).

FIGS. 7A-B illustrate the rotational movement of the finger levers of the present controller (FIG. 7A) and the corresponding rotation of a tissue manipulator end of a surgical device (FIG. 7B).

FIGS. 8A-D illustrate the up-down movement of fingers interface portion of the present controller (FIGS. 8A, C) and the corresponding up-down deflection of a steerable shaft of a surgical device (FIGS. 8B, D).

FIGS. 9A-D illustrate the side-to-side movement of fingers interface portion of the present controller (FIGS. 9A, C) and the corresponding side-to-side deflection of a steerable shaft of a surgical device (FIGS. 9B, D).

FIGS. 10A-B illustrate the angular movement sensors of the first portion of the present controller.

FIGS. 11A-B illustrate the linear movement sensors of the second portion of the present controller.

FIGS. 12A-B illustrate the angular movement sensors of the third portion of the present controller.

FIG. 12C illustrates a button enabling a user to switch control between several surgical tools.

FIGS. 13A-C illustrate various mounting configurations of the present controller.

FIGS. 14A-C are photos of a prototype controller constructed according to the teachings of the present invention.

### DESCRIPTION OF THE PREFERRED EMBODIMENTS

The present invention is of a controller which can be used to locally or remotely control the operation of one or more surgical tools including endoscopes, laparoscopes and robotic tool systems. Specifically, the present invention can be directly or wirelessly (or through communication network) attached to surgical tools to control the operation thereof or used remotely (within or outside the operating theater) to control robotic surgical systems.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Controllers for surgical tools are well known in the art and are used for controlling mechanical, motorized or robotic tools. Such controllers can be used to accurately position and control surgical instruments within the body, however, they can be bulky and difficult to operate and oftentimes require a long training period to master.

While reducing the present invention to practice, the present inventor set out to design a surgical tool controller that can be used to easily and naturally control one or more surgical tools. The present controller was designed to translate the natural movements of the users arm, hand and fingers to a specific motion and actuation of the surgical tool. This enables a user to move and orient the hand naturally without having to pay attention to movement of specific parts of the controller. In other words, the user does not need to separately control each portion of the controller in order to effect movement of the surgical tool but rather employs one fluid and coordinated movement of the arm, hand and fingers in order to position and actuate the surgical tool. Since the present controller includes several portions arranged lengthwise, with each being independently movable in one or more axis, any complex movement produced by a human arm, hand and fingers can be accurately tracked by the present controller and translated into similarly complex movement in one or more surgical instruments.

The controller of the present invention was specifically designed to provide the following: (i) to translate natural movement of the user's hand and fingers into accurate movements of one or more surgical instruments.

(ii) to be quickly mastered by a user.

(iii) to provide a compact and light weight interface that can be carried by a user

(iv) to negate the use of foot pedals

(v) to provide universal control over any type of surgical tool

Thus, according to one aspect of the present invention there is provided a controller for a surgical tool.

As used herein the phrase “surgical tool” refers to any tool used in a surgical procedure (open or minimal) to manipulate, view or otherwise assist in the procedure. Examples of a surgical tool include, but are not limited to, an endoscope (e.g. gastroscope, colonoscope, laparoscope) having an effector end such as a grasper, a needle, a camera, suction, a diathermia hook or bi-polar grasper. The endoscope can include a rigid, flexible or steerable shaft terminating with one or more effector ends.

Endoscopic tools are delivered through a small diameter delivery port (e.g. trocar) and are utilized in an anatomically constrained space thus, an endoscope having a steerable shaft that can be deflected inside the body using controls positioned outside the body can be advantageous for use. Such steering enables an operator to guide the endoscope within the body and accurately position a distally-mounted effector at an anatomical landmark. Steerable tools typically employ one or more control wires which run the length of the shaft and terminate at the distal end of the steerable portion or at the distal tip.

The controller of the present invention includes an elongated body having interconnected first, second and third portions. The first portion includes a proximal end attachable to a support (e.g. chair, bed, belt of a user) and a distal end connected to the second portion. Such a connection allows the second portion to move with respect to the first portion. The third portion is connected to the second portion through a connecter configured for enabling the third portion to move with respect to the second portion. The third portion includes controls engageable via a hand and/or fingers of the user.

Although the present controller is preferably utilized with motorized surgical tools and is functionally attached thereto via wired or wireless interfaces, a configuration in which the controller includes a proximal end designed for directly (and mechanically) interfacing with a control unit of a non-motorized surgical tool is also envisaged herein.

Thus, the present controller includes three interconnected and independently movable portions each being capable of controlling a different function of a surgical tool functionally linked to the present controller.

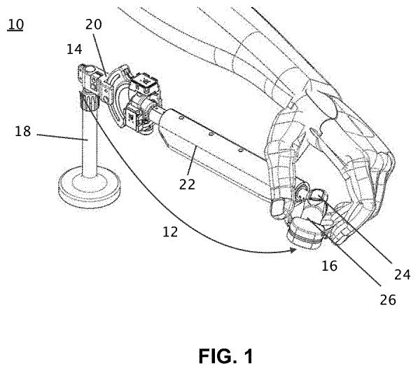

Referring now to the drawings, FIG. 1 illustrates the present controller which is referred to herein as controller 10.

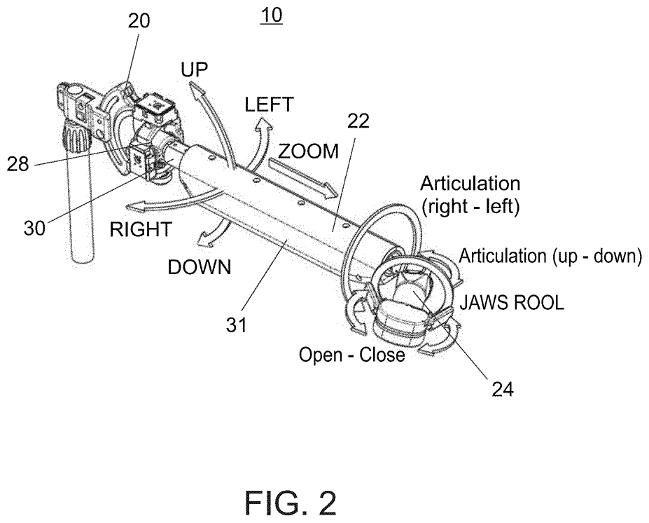

Controller 10 includes an elongated body 12 having a proximal end 14 and a distal end 16. In the configuration shown in FIGS. 1-2, proximal end 14 is connected to a support post 18. Such a support post 18 can be attached to a chair, a bed or any stable structure. Alternatively, support post 18 can be mounted on a belt of a user.

Elongated body 12 includes three portions, a first portion 20, a second portion 22 and a third portion 24. Third portion 24 includes hand/fingers interface 26 shown grasped by a user's fingers in FIG. 1.

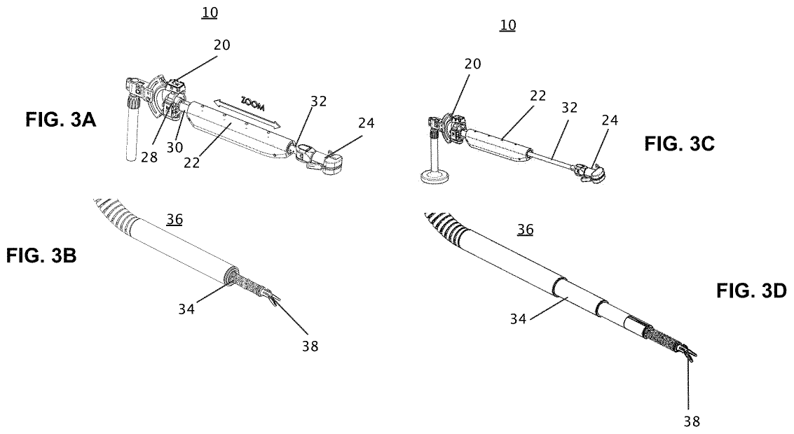

FIG. 2 illustrates the movements of each of portions 20, 22 and 24. Portion 20 includes a gimbal-like joint 28 that enables up-down, left-right movement of portions 22 and 24 of elongated body 12. Portion 22 is connected to gimbal joint 28 of portion 20 through connector 30. Portion 22 includes cover 31 serving as housing for the linear sensor that measures the rail 32 (FIGS. 3A and C) movement.

Portion 24 is mounted on a rail 32 (FIGS. 3A and C) that can be moved in and out of portion 22 (zoom in-out). Portion 24 can also be rolled together with portion 22 with respect to portion 20 while hand/fingers 26 can be moved up-down and side-to-side with respect to portion 22.

Elongated body can be 100-300 mm in length and 10-30 mm in diameter. The linear movement range of elongated body (the delta between fully retracted and fully expanded states) can be 50-250 mm. Portion 22 can be angled up/down range ±60 degrees and right/left ±90 degrees. Portion 24 can be angled up/down right/left ±90 degrees and rolled right/left ±90 degrees. Finger interface 26 can be rolled ±30 degrees and levers 40 can open/close ±30 degrees.

FIGS. 3A-9B illustrate various movements of controller 10 and the corresponding movements of a surgical tool.

FIGS. 3A-D illustrate the ‘zoom’ function of controller 10. Retraction and extension of portion 24 with respect to portion 22 (as is shown in FIGS. 3A and C respectively) retracts and extends a telescopic shaft 34 of a surgical tool 36. Such movement allows a user to extend/retract shaft 34 within the body to better position an effector end 38 at a tissue site. The linear movement range of portion 24 is typically 0-200 mm.

The ratio of movement between portion 24 and shaft 34 can be 1:1 (absolute control) or it can be 2:1, 3:1 etc. or vice versa (relative control).

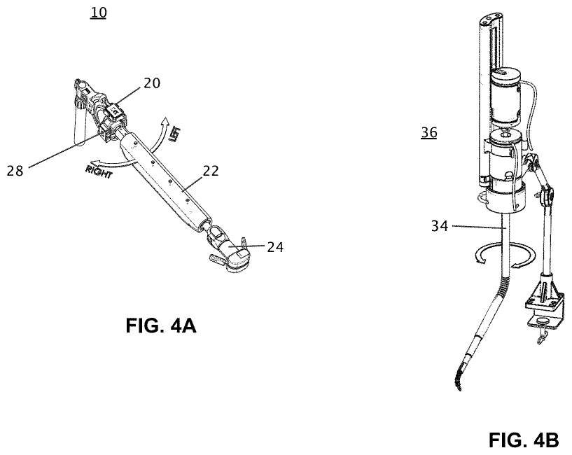

FIGS. 4A-B illustrate side-to-side (left-right) movement (FIG. 4A) of portions 22 and 24 with respect to portion 20 (through gimbal joint 28) and the corresponding left-right rotation (FIG. 4B) of steerable shaft 34 of surgical tool 36. Both relative and absolute control can be used with this tool movement.

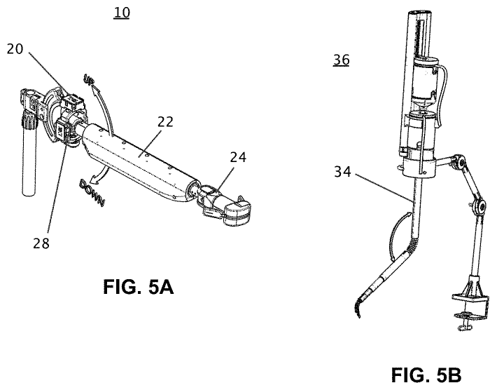

FIGS. 5A-B illustrate up-down movement of portions 22 and 24 (FIG. 5A) with respect to portion 20 (through gimbal joint 28) and the corresponding up-down deflection of steerable shaft 34 of surgical tool 36 (FIG. 5B). Both relative and absolute control can be used with this tool movement.

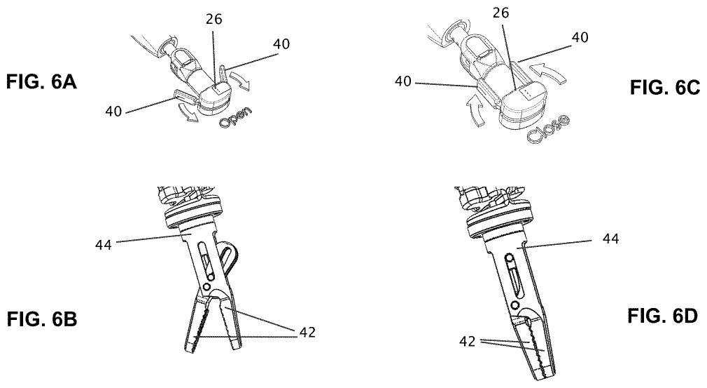

FIGS. 6A-D illustrate movement of finger levers 40 of interface 26. Angular opening of levers 40 (FIG. 6A) opens jaws 42 (FIG. 6B) of a grasper 44 (attached to a distal end of a surgical tool shaft), while angular closing of levers 40 (FIG. 6C) closes jaws 42 of grasper 44 (FIG. 6D). Fingers levers 40 open/close ±30 degrees. Relative control at scale of 1:2 of angular movement (Lever: Jaw) can be used between movement of finger levers 40 and jaws 42 of grasper 44.

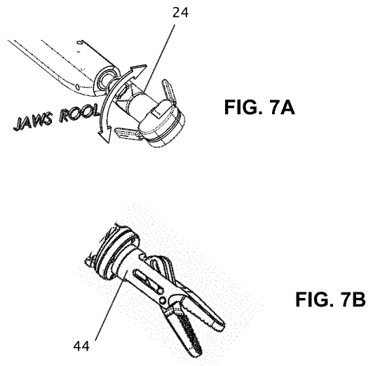

FIGS. 7A-B illustrate rotation of fingers segment of portion 24, (FIG. 7A) and the corresponding rotation of grasper 44 (FIG. 7B). Relative control at a scale of up to 1:7 of angular movement (finger levers: Jaw) can be used between rotation of portion 24 and that of grasper 44.

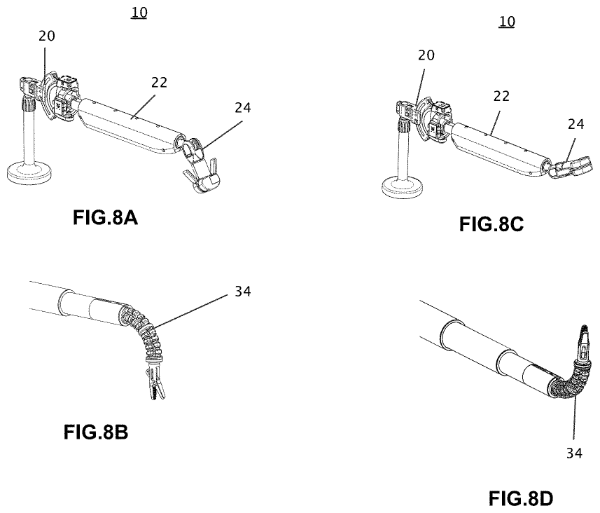

FIGS. 8A-D illustrate up-down movement of portion 24 with respect to portion 22 (FIGS. 8A and C respectively) and the corresponding up-down deflection of distal steerable part of shaft 34 (FIGS. 8B and D respectively). Both relative and absolute control can be used between movement of portion 24 and that of distal steerable part of shaft 34.

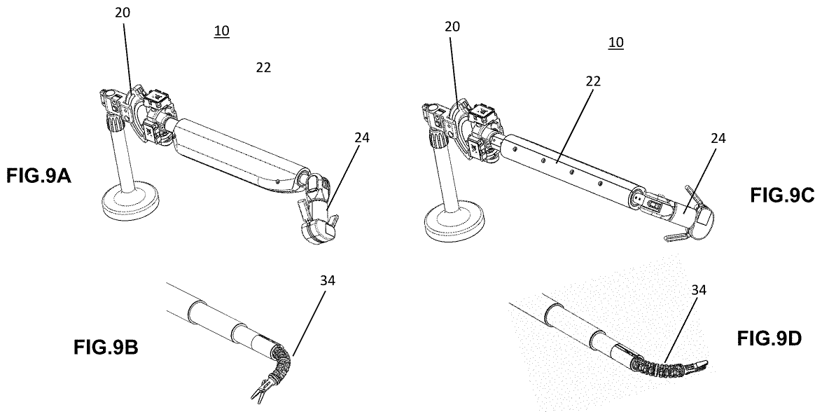

FIGS. 9A-D illustrate side-to-side tilt (roll) of portion 24 together with portion 22, with respect to portion 20 (FIGS. 9A and C) and the corresponding right-left deflection of distal steerable part of shaft 34 (FIGS. 9B and D). Both relative and absolute control can be used between movement of portion 24 and that of distal steerable part of shaft 34.

Controller 10 can be physically connected to a surgical tool, alternatively, controller 10 can be on the surgeon belt or connected via tripod to the surgeon seat or to the patient bed. The communication between the controller to the motorized surgical tools may be by physical wire or wirelessly connected (via RF/infra-red/light communication) to control one or more motors that actuate movement of the shaft, effector end etc.

In the latter configuration, controller 10 includes several sensors positioned along elongated body 12 that measure relative movement between portions 20, 22 and 24, as well as finger levers 40.

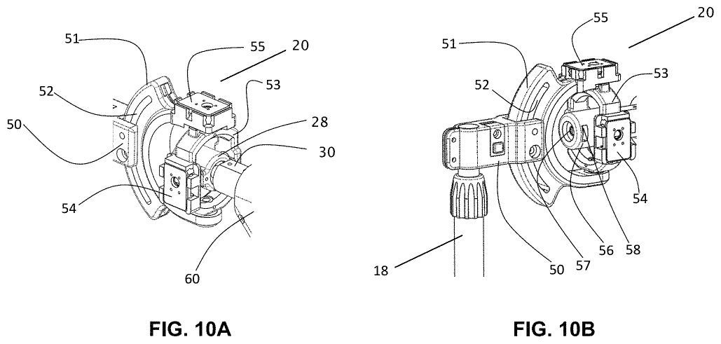

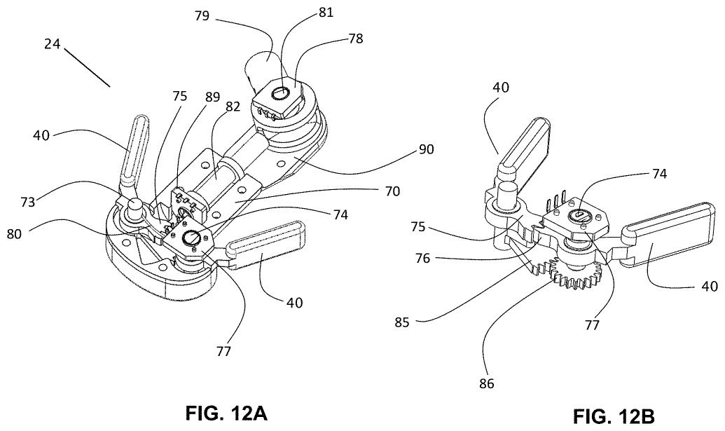

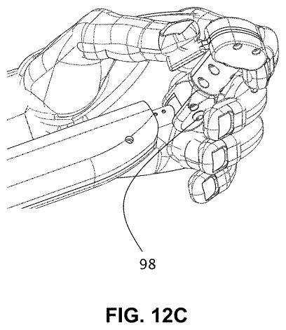

FIGS. 10A-12B illustrate sensor arrangements for portions 20, 22 and 24 (FIGS. 10A-B, 11A-B and 12A-B respectively) as well as fingers levers 40 (FIGS. 12A-B).

FIGS. 10A-B illustrate portion 20 of controller 10. The proximal end of portion 20 is connected to support post 18 through clamp 50. The distal end of portion 20 is rotatably connected to portion 22 through joint 28. Clamp 50 is connected to outer gimbal arc 50 via slot 52. Slot 52 allows to the surgeon to rotate gimbal arc 50 with respect to support post 18, enabling the surgeon to adjust the orientation of portion 20. Gimbal ring 53 is rotatably connected to outer gimbal 51. Rotation sensor 55 is connected to gimbal 53 shaft and measures the right/left movement of portion 22. Inner cylinder 56 is rotatably connected to ring 53. The rotation axis of cylinder 56 are perpendicular to the shafts connecting ring 53 to outer arc 51. Rotation sensor 54 is connected to the shaft of cylinder 56 and measures the up/down movement of portion 22.

Shaft 30 is rotatably connected to cylinder 56. Rotation sensor 58 is connected to shaft 30 and measures the tilt (roll) movement of portion 24 together with portion 22.

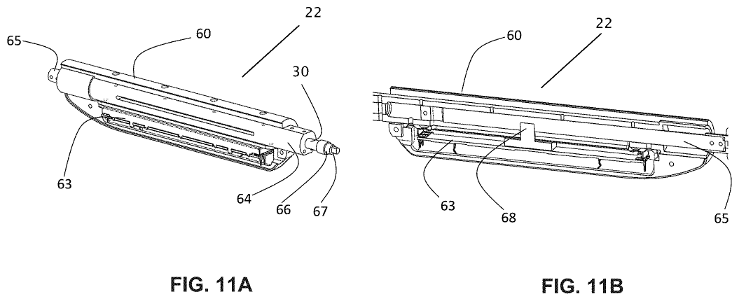

FIGS. 11A-B illustrate portion 22 of controller 10. Shaft 30 is shown to the right at the proximal end of portion 22. As is described above, shaft 30 is connected to a rotation measurement sensor 58, located in central cylinder 56 with shaped axis end 67. Rotation of portion 22 (via wrist tilting of fingers portion 24) co-rotates shaft 30 and the degree of fingers tilt is measured via rotation sensor 58.

Sensor 63 measures the linear movement of shaft 65. Shaft 65 is telescopically mounted in hollow body 64 of portion 22. When shaft 65 is moved back and forth relative to body 64, it carries slider 68, a linear sensor 63 measures the position of slider 68 relative to portion 22. Cover 60 serves as a housing for linear sensor 63, body 64 and shaft 30. Shaft 65, connects portion 22 to portion 24.

The sensors described herein can be electric linear potentiometers such as Linear Type RDC10 Series by ALPS, magnetic Hall Effect sensors such as LX90393SLW-ABA-011-RE by Melexis Technologies NV, or multi rotational potentiometers such as 35905-2-103L by Bourns Inc.

To operate controller 10, a user grasps portion 24 and moves portions 22 and 24 to a desired spatial position (up/down, rotate, side to side, forward backwards). The movement of controller 10 is mimicked by movement of the surgical tool(s) controlled thereby. The user can also simultaneously control an effector end (e.g. grasper) via levers 40. Portion 24 can also be angled and rotated with respect to portion 20. Actuation of levers and movement of portion 24 can be affected simultaneously with or independently of movement of other portions.

Shaft 79 of portion 24 is fixed to distal shaft 65 of portion 22. Body 90 is connected to shaft 79 through shaft 81. Body 90 can rotate around shaft 81 with rotation measured via sensor 78 that is fixed to body 90; shaft 81 runs through the rotating part of sensor 78. When body 90 rotates, rotation sensor measures up/down movement of portion 24.

Body 70 is connected to body 90 via shaft 82 and is rotatable around shaft 82 under finger control. Rotation sensor 79 is fixed to body 70 with shaft 82 running through rotation sensor 89. When body 70 rotates, rotation sensor 79 measures roll movement of portion 24.

Levers 40 are located at the distal end of body 70 and rotate around shafts 73, and 74. Levers 40 are interconnected through gears 75 and 76 to ensure identical movement of levers 40 while allowing the user to open and close levers 40 by applying a force to only one of levers.

Gear 85 (a part lever 40) meshes with gear 86 which is rigidly connected to shaft 74 which in turn runs through rotation sensor 77. When shaft 74 rotates, rotation sensor 79 measures the angular movement of levers 40.

Gears 85 and 86 may be of equal or different diameters. This allows to select different sensitivity's to the open/close action. Spring 80 connects shafts 73 and 74. When a closing force is applied to levers 40, spring 80 applies a counter opening force to thereby provide a user with better sensitivity of lever 40 travel and enable levers to automatically open when the closing force is released.

As is mentioned hereinabove, controller 10 can be mounted on a fixture (tripod, chair, bed) or directly on a user (via use of a belt or harness).

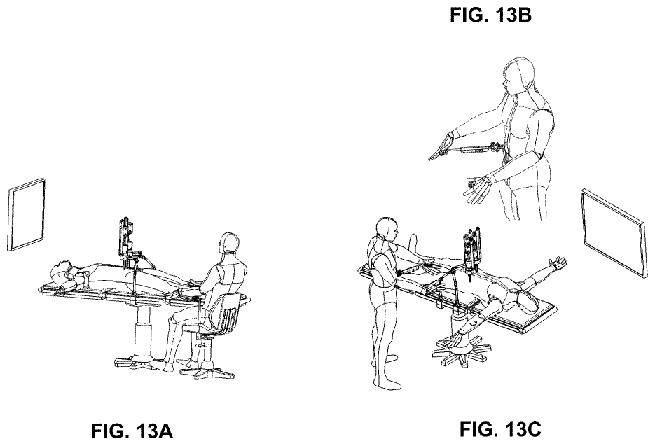

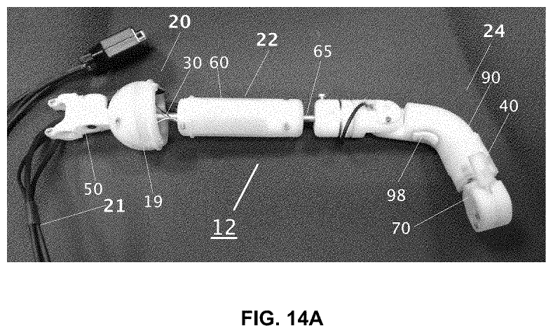

FIGS. 13A-C illustrate mounting of controller 10 on a bed frame 80 (FIG. 13A) and user 82 (FIGS. 13A-B).

FIG. 13A illustrates a configuration in which a user (e.g. surgeon) is sitting on a chair with at least one controller 10 attached to the chair. Elongated body 12 of controller 10 is typically located under and along the surgeon forearm with portion 20 located under the elbow of the surgeon and portion 24 at the surgeon's fingers. In this configuration, controller 10 can be effortlessly and naturally moved by the surgeon while viewing the anatomical site through a video screen connected to laparoscopic camera.

FIG. 13B illustrate a configuration were at least one controller 10 is attached to the surgeon through a belt. Elongated body 12 of controller 10 is typically located under and along the surgeon forearm with portion 20 located under the elbow of the surgeon and portion 24 at the surgeon's fingers. In this configuration, controller 10 can be effortlessly and naturally moved by the surgeon while viewing the anatomical site through a video screen connected to laparoscopic camera and being free to move in the operating room as shown in FIG. 13C.

FIG. 13C illustrates a 2 controller 10 setup attached to the surgeon belt. The surgeon stands near the patient bed and operates while viewing the anatomical site through a video screen. One of the advantages of this configuration is that the surgeon is close the patient.

One or more controllers 10 of the present invention can be utilized in any type of minimally invasive or fully open procedure. The following describes use of controller 10 in controlling laparoscopic tools in a minimally invasive surgical procedure.

Several incisions are made in a tissue wall to create several access sites. Each site is then used to position a trocar through which a surgical tool (grasper, cutter, camera) is advanced until the effector end is positioned within the body cavity.

The surgical tools can be robotic tools with a motor pack connected to a [proximal (extracorporeal) end thereof). One or more controllers 10 are mounted on a fixture and/or surgeon and the controls are tested in order to ensure that the movements of the controller(s) are correctly oriented with the movements of the surgical tool. If one or more surgical tools are not correctly oriented, the surgeon can manually or automatically (through motor) rotate the surgical tool(s) to the correct orientation. When the orientations are set, the controller(s) is ready for the procedure.

If the procedure calls for a setup in which surgical tools must be in different orientations, for example when one surgical tool (e.g. grasper) is oriented with the surgeon and another tool (e.g. camera) is oriented from an opposite side, then the surgeon can setup each controller for such a setup.

The surgeon can use two or more controllers 10 to control two or more surgical tools. Alternatively, the surgeon can use a single controller 10 to sequentially control two or more surgical tools. Controller 10 can include a dialog button 98 (FIG. 12C) to allow switching between tools. Dialog button 98 controls communication between controller 10 and a surgical tool. When depressed, this button can link or free controller 10 from the surgical tool or allow switching between several tools. When a user switches between tools, the freed tool stays in the last controlled position. When returning to this tool, the surgeon need not reorient controller 10 to match the ‘paused’ position of this tool. Such a relative control approach allows the surgeon to disengage (let go) the controller and reengage it after choosing a more comfortable arm position and proceed with the procedure without having to match tool position with controller position prior to disengagement.

Relative control also enables switching control between any number of surgeons with the surgeon activating the dialog button assuming control over a surgical tool. Such transfer of control can be seamless since the spatial positioning of the surgeon's controller does not need to match that of the surgical tool.

Under absolute control, the surgeon has to match controller position with that of the tool. Software with safety algorithm can compensate for the differences by applying smooth and filtered paths of movements bridging the movement from one controller position to another.

Thus, the present invention provides a compact and light controller that can be positioned anywhere or carried by the user. While the present controller is light and small it can follow the most complex movements of the human hand in 6 axis. This is achieved by a jointed interface having sensor mechanisms for detecting both large (cm) and small (micron) range movements of one controller portion with respect to another.

While a typical console-type interfaces/controller positions the controls in front of the surgeon, the present controller needs only to be placed in the same general orientation of the surgical tool. The proximal end of the controller is typically placed near the elbow of the surgeon and the distal end near the surgeons fingers.

The present controller enables relative control over a surgical tool thereby allowing the surgeon to choose the most ergonomic position for operating the controller even mid procedure. An added benefit of relative control is that it allows the controller to be light and compact since a large range of movement can be effected using a series of small movements interrupted by controller repositioning.

Any number of the present controller can be used simultaneously by one or more users to control any number of surgical instruments.

As used herein the term “about” refers to ±10%.

Additional objects, advantages, and novel features of the present invention will become apparent to one ordinarily skilled in the art upon examination of the following examples, which are not intended to be limiting.

### EXAMPLES

Reference is now made to the following example, which together with the above descriptions, illustrate the invention in a non-limiting fashion.

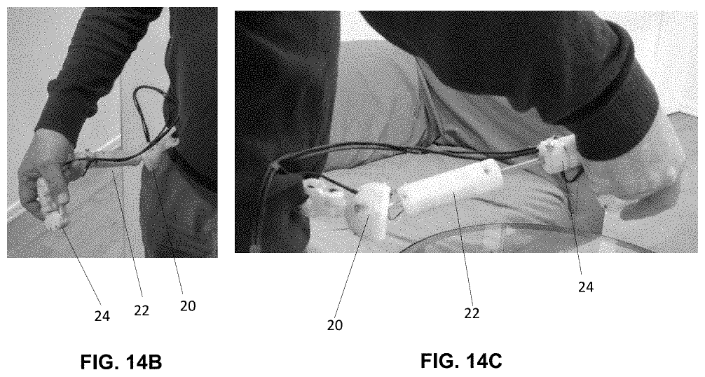

A prototype controller constructed according to the teachings of the present invention was tested for operability (FIGS. 14A-C).

The body of the prototype controller was manufactured from polyamide using 3D printing approaches and shafts 65 and 30 were fabricated from stainless steel (FIG. 14A). The prototype is 200 mm long when portion 22 is retracted (FIG. 14B) and 250 mm in length when telescopic shaft 65 is extended (FIG. 14C). The diameter of cover 60 of portion 22 is 30 mm. A Dialog button 98 is positioned at the lower portion of segment 90 and finger levers 40 extend from the distal end of segment 70.

The inner parts of portion 20 are covered via sphere 19. A communication cable 21 connects the controller to control circuits of one or more robotic (motorized) surgical instruments.

FIGS. 14B-C show a photo of the controller prototype attached at a user's torso region (clamped to clothing).

The user holds the controller at distal segment 24 with fingers positioned on levers 40 and dialog button 98 (FIG. 14B).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A controller for a surgical tool comprising an elongated body having:
(a) a first portion having a proximal end attachable to a support and a distal end connected to a second portion through a first connector configured for enabling said second portion to move with respect to said first portion; and
(b) a third portion connected to said second portion through a second connecter configured for enabling said third portion to move with respect to said second portion, said third portion having an interface engageable by a hand and/or fingers of a user.

2. The controller of claim 1, wherein said proximal end of said first portion is movable with respect to said support when attached thereto.

3. The controller of claim 2, wherein said first portion includes a first sensor for measuring a movement of said first portion with respect to said support.

4. The controller of claim 1, wherein said elongated body includes a second sensor for measuring a movement of said second portion with respect to said first portion.

5. The controller of claim 1, wherein said elongated body includes a third sensor for measuring a movement of said third portion with respect to said second portion.

6. The controller of claim 1, wherein said elongated body is positioned under a forearm of a user when said interface of said third portion is engaged by hand and/or fingers of said user.

7. The controller of claim 1, wherein said first portion is attachable to said support frame through a pivot.

8. The controller of claim 2, wherein said first portion is capable of rolling and/or pivoting with respect to said support.

9. The controller of claim 1, wherein said second portion is capable of translating and/or rolling with respect to said first portion.

10. The controller of claim 1, wherein said third portion is capable of translating rolling and/or pivoting with respect to said second portion.

11. The controller of claim 1, wherein said interface include levers engageable by a thumb and forefinger of said user.

12. The controller of claim 11, wherein said second portion is engageable by a palm of said user.

13. The controller of claim 1, further comprising a wireless transceiver for communicating with a surgical tool.

14. A system comprising the controller of claim 1 attached to a surgical tool.

15. The system of claim 14, wherein said surgical tool is an endoscope.

16. The system of claim 14, wherein said surgical tool is steerable and includes an effector end.

17. The system of claim 16, wherein said effector end is a grasper, scissors, a needle, a camera, suction or a clamp.
